(19) United States
(12) Patent Application Publication
(10) Pub. No.: US 2011/0306127 A1
(43) Pub. Date: Dec. 15, 2011

(54) ENHANCING IRON UPTAKE IN PROTEIN-FREE MEDIA

(75) Inventors: Seth Adrian Miller, Englewood, CO (US); Russell C. Marians, Englewood, CO (US)

(73) Assignee: Empire Technology Development LLC

(21) Appl. No.: 13/063,973

(22) PCT Filed: Jun. 10, 2010
(86) PCT No.: PCT/US10/38100

§ 371 Date: Mar. 15, 2011

(51) Int. Cl. C12N 5/071; C12N 5/09; C12N 5/077; C12N 5/078; C12N 5/0786

(52) U.S. Cl. 435/358; 435/375; 435/366; 435/372; 435/370; 435/369; 435/367; 435/404

(57) ABSTRACT

Methods for enhancing iron uptake in cell culture are described. The methods include using a NTBI uptake activator and non-protein bound iron source. Suitable NTBI activators are delineated. Also described are serum-free culture media which can be used in the present methods.

### TECHNICAL FIELD

The present technology relates generally to cell culture media and to materials and methods for enhancing iron uptake in cell culture.

### BACKGROUND

The following description is provided to assist the understanding of the reader. None of the information provided or references cited is admitted to be prior art.

Iron is an essential metal in cell culture. Most eukaryotic cell culture systems use transferrin (Tf), a serum protein, as a primary staple iron source/transporter. It is so indispensable for these culture systems that it is frequently—and incorrectly—referred to as a “growth factor.” Eukaryotic transferrins comprise a class of bi-lobal iron-binding proteins, each lobe bearing a single site capable of reversibly binding iron and accounting for the physiological roles of the proteins in iron transport and iron withholding as a defense against infection. Tf normally provides iron for cellular needs and for most cells, the delivery of transferrin-borne iron depends on association of the protein with transferrin receptors, TfR1 and TfR2, on plasma membranes. An elaborate receptor-mediated pathway drives endocytosis of Tf-bound iron into mammalian cells for use and storage. TfR1 and TfR2 play critical roles in iron transfer involving transferrin. Transferrin is derived from animal-derived serum—causing sourcing, contamination, and quality assurance problems, among many others—or through transgenic production making it an expensive additive. Therefore, use of transferrin and other protein growth factors in cell media can be problematic because of their higher cost, the potential to carry over harmful comtaminants such as viruses or prions to clean media, and the need to purify the biologic away from the relatively more abundant media proteins. Consequently, protein-free media are desirable for large scale cell culture and production of biologics.

Growth, metabolism and maintenance of cells requires iron as an essential nutrient. It has both beneficial and toxic properties. Consequently, the management of iron levels and delivery are a major challenge. Mammalian cells accumulate iron from two main circulating sources. The first one, which is a classical source, consists of iron bound to transferrin, as described above, and the second one is called Non-Transferrin-Bound Iron (NTBI). NTBI uptake involves transport of the metal directly across the cell surface in the absence of Tf, and does not depend on endocytosis. This is particularly useful for applications in protein-free cell culture. Protein-free media may be suitable for large scale cell culture and production of biologics. NTBI is typically weakly complexed to albumin, citrate, amino acids, sugars and other small molecules. Iron transport across the cell membrane can take place in both directions. For iron deficient patients, for example, an effective transport of iron from external sources into the cells is required. This requirement is complicated by the fact that environmental iron is invariably present as insoluble Fe³⁺ leading to poor bioavailability and toxicity, since even low concentrations of iron catalyze the production of damaging reactive oxygen species. As a result, activators which provide efficient uptake and transport systems to extract iron from their environment as well as ferritins that store iron in a non-toxic form are required. On the other hand, excess iron in the cells has been reported to be associated with several clinical states such as thalassemia, hemochromatosis, and in patients receiving chemotherapy. In such cases, NTBI can be highly toxic to cells as it can potentiate the formation of free radicals through Fenton reaction and thereby induce cell damage. Hence, an effective iron uptake agent can also be employed to remove NTBI from cells. A few ligands have been evaluated as potential chelators or iron-binding agents to capture NTBI without chelating transferrin- or ferritin-bound iron in plasma. But many of these ligands bind too tightly to the iron metal and fail to unload the metal once it reaches the cell. On the other hand, activators which form a very weak complex with iron are also not desirable because free or ineffectively sequestered iron can be very toxic to cells. Suitable NTBI transport activators are therefore needed which bind tight enough to allow transport across the cell membrane but bind loose enough to unload the metal after it crosses the cell membrane and thus enhance facilitate transport of NTBI across the cell membrane from the medium to the cells.

Cells cultivated in culture media catabolize available nutrients and produce useful biological substances such as viruses, monoclonal antibodies, hormones, growth factors and the like. Such products have therapeutic applications and, with the advent of recombinant DNA technology, cells can be engineered to produce large quantities of many of these products. Mammalian cell culture is used in many recombinant protein production processes due to its ability to produce proteins with proper post-translational modifications. Thus, the ability to cultivate cells in vitro is not only important for the study of cell physiology, but is also necessary for the production of useful substances which may not otherwise be obtained using cost-effective production.

Cell culture media formulations have been well documented in the literature and a number of media are commercially available. Cell culture media provide the nutrients necessary to maintain and grow cells in a controlled artificial environment. Characteristics and compositions of the cell culture media vary depending on the particular cellular requirements. Important parameters include osmolarity, pH, and nutrient formulations. The requirements of mammalian cell culture in vitro include, in addition to basic nutritional substances, a complex series of growth factors. Usually, these are added to the culture medium by supplying it with animal sera or protein fractions from animal sources. However, these chemically non-defined mixtures exhibit variable lot to lot composition. Such mixtures also represent a potential source of contaminants including viruses and mycoplasmas. For production on an industrial scale, the high price of the supplements and difficulties in downstream processing are additional considerations.

### SUMMARY

In accordance with one aspect, the present technology relates to a method for enhancing iron uptake in cell culture. In one embodiment, the present disclosure provides a method for enhancing iron uptake in a mammalian cell culture, the method comprising: contacting cells with a culture medium containing a NTBI uptake activator and a non-protein-bound iron source, wherein the NTBI uptake activator is present in an effective amount to enhance the iron uptake of the cells cultured therein compared to cells not contacted with the NTBI uptake activator.

In some embodiments, the NTBI uptake activator is not a calcium channel blocker. In other embodiments, the NTBI uptake activator is a nitrogen heterocycle. In some embodiments, the nitrogen heterocycle is a pyridine or pyrazine. In certain embodiments, the pyridine is a substituted pyridine. In an illustrative embodiment, the substituted pyridine is a 2,6 substituted pyridine.

In some embodiments, the NTBI uptake activator is a nitrogen heterocycle, wherein the nitrogen heterocycle is a compound having formula I:

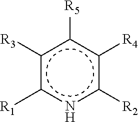

wherein
each of R₁ and R₂ is selected from substituted or unsubstituted alkyl, substituted or unsubstituted aryl, substituted or unsubstituted aralkyl, substituted or unsubstituted alkylamino, substituted or unsubstituted arylamino, substituted or unsubstituted heterocyclyl, and substituted or unsubstituted heteroaryl;
each of R₃, R₄ and R₅ independently represents hydrogen, halogen, hydroxyl, oxo, nitro, nitrile, amino, COOR′, substituted or unsubstituted alkyl, substituted or unsubstituted alkoxy, substituted or unsubstituted aryl, substituted or unsubstituted aralkyl, or substituted or unsubstituted heterocyclyl; and
R′ is a hydrogen or an alkyl group.

In some embodiments, the nitrogen heterocycle is a compound having formula Ia:

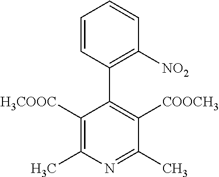

In some embodiments, the nitrogen heterocycle is a compound having formula II:

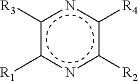

wherein
each of R₁, R₂, R₃ and R₄ independently represents hydrogen, halogen, hydroxyl, oxo, nitro, nitrile, amino, COOR′, substituted or unsubstituted alkyl, substituted or unsubstituted alkoxy, substituted or unsubstituted aryl, substituted or unsubstituted aralkyl, substituted or unsubstituted alkylamino, substituted or unsubstituted arylamino, substituted or unsubstituted heterocyclyl, or substituted or unsubstituted heteroaryl; and
R′ is a hydrogen or an alkyl group.

In some embodiments, the nitrogen heterocycle is present in the culture medium in a final concentration of about 0.5 μM to about 200 μM. In some embodiments, the non-protein-bound iron source is ferrous sulfate. In some embodiments, the non-protein-bound iron source is an iron-organic ion chelate. In some embodiments, the iron-organic ion chelate is ferric ammonium citrate. In some embodiments, the non-protein iron source is present in the culture medium in a final concentration of about 0.4 μM to about 100 μM.

In some embodiments, the cells are human cells. In some embodiments, the human cells are selected from the group consisting of lymphocytes, myeloid cells, monocytes, macrophages, neutrophils, myocytes, fibroblasts, HepG2 carcinoma cells, kidney cells, melanoma cells, and HeLa cells. In some embodiments, the cells are non-human mammalian cells. In some embodiments, the non-human mammalian cells are Chinese hamster ovary cells. In some embodiments, the culture medium lacks transferrin. In some embodiments, the culture medium is a serum-free media.

In another aspect, the technology provides a serum free media for mammalian cell culture. In some embodiments, the serum free medium comprises a nitrogen heterocycle and a non-protein-bound iron source, wherein the nitrogen heterocycle is present in an effective amount to enhance the iron uptake of the cells cultured therein compared to cells not contacted with the nitrogen heterocycle.

In yet another aspect, a kit for enhancing iron uptake in a mammalian cell culture comprising a nitrogen heterocycle and a non-protein-bound iron source is provided. In some embodiments of the kit, the nitrogen heterocycle is present in an effective amount to enhance the iron uptake of the cells cultured therein compared to cells not contacted with the nitrogen heterocycle.

The foregoing summary is illustrative only and is not intended to be in any way limiting. In addition to the illustrative aspects, embodiments, and features described above, further aspects, embodiments, and features will become apparent by reference to the following drawings and the detailed description.

### DETAILED DESCRIPTION

In the following detailed description, the illustrative embodiments described in the detailed description, figures, and claims are not meant to be limiting. Other embodiments may be utilized, and other changes may be made, without departing from the spirit or scope of the subject matter presented here. In the description that follows, a number of terms are used extensively. The terms described below are more fully understood by reference to the specification as a whole. Units, prefixes, and symbols may be denoted in their accepted SI form.

The terms “a” and “an” as used herein mean “one or more” unless the singular is expressly specified. Thus, for example, reference to “a cell” includes a mixture of two or more cells, as well as a single cell.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to, plus or minus 10% of the particular term.

As used herein, the term “activator of iron uptake” or “iron uptake activator” refers to a compound that activates a non-transferrin bound iron (NTBI) transport pathway.

As used herein, the term “cytokine” refers to a compound that induces a physiological response in a cell, such as growth, differentiation, senescence, apoptosis, cytotoxicity or antibody secretion. Included in this definition of “cytokine” are growth factors, interleukins, colony-stimulating factors, interferons, lymphokines and the like.

As used herein, the term “cell culture” or “culture” is meant the maintenance, growth and proliferation of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” or “organ system culture” may occasionally be used interchangeably with the term “cell culture.” The media described herein can be used to culture any mammalian cell.

As used herein, the term “cultivation” is meant the maintenance of cells in vitro under conditions favoring growth, differentiation or continued viability, in an active or quiescent state, of the cells. In this sense, “cultivation” may be used interchangeably with “cell culture” or any of its synonyms described above.

As used herein, the term “culture vessel” is meant a glass, plastic, or metal container that can provide an aseptic environment for culturing cells.

As used herein, the term phrases “cell culture medium,” “culture medium” (plural “media” in each case) and “medium formulation” refer to a nutritive solution for cultivating cells and may be used interchangeably.

As used herein, the term “contacting” refers to the placing of cells to be cultivated in vitro into a culture vessel with the medium in which the cells are to be cultivated. The term “contacting” encompasses mixing cells with medium, pipetting medium onto cells in a culture vessel, and submerging cells in culture medium.

As used herein, the term “combining” refers to the mixing or admixing of ingredients in a cell culture medium formulation.

As used herein, a “chemically defined” medium is one for which every ingredient is known. A chemically defined medium is distinguished from serum, embryonic extracts, and hydrolysates, each of which contain unknown components. The medium of the present technology is chemically defined and is free of proteins and peptides.

As used herein, the term “ingredient” refers to any compound, whether of chemical or biological origin, that can be used in cell culture media, to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

As used herein, a “protein-free” medium is one which contains no proteins or peptides. A protein-free medium is distinguished from low-protein and essentially protein-free media, both of which contain proteins and/or peptides.

The term “transport,” as in the “transport” of a compound of interest across a cell membrane, refers to passage of the compound in the direction of external to internal movement.

The terms “optional” and “optionally” mean that the subsequently described circumstance may or may not occur, so that the description includes instances where the circumstance occurs and instances where it does not.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the technology. Procedures for inserting such labels into the compounds of the technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments, the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include, but are not limited to, benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups.” Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl(pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl(azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl(azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Groups described herein having two or more points of attachment (i.e., divalent, trivalent, or polyvalent) within the compound of the technology are designated by use of the suffix, “ene.” For example, divalent alkyl groups are alkylene groups, divalent aryl groups are arylene groups, divalent heteroaryl groups are heteroarylene groups, and so forth. Substituted groups having a single point of attachment to the compound of the technology are not referred to using the “ene” designation. Thus, for example, chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include, but are not limited to, methoxy, ethoxy, propoxy, butoxy, pentoxy, hexoxy, and the like. Examples of branched alkoxy groups include, but are not limited to, isopropoxy, sec-butoxy, tert-butoxy, isopentoxy, isohexoxy, and the like. Examples of cycloalkoxy groups include, but are not limited to, cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include, but are not limited to, phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR³⁰ groups. R³⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR³¹R³², and —NR³¹C(O)R³² groups, respectively. R³¹ and R³² are independently hydrogen, or a substituted or unsubstituted alkyl, alkenyl, alkynyl, cycloalkyl, aryl, aralkyl, heterocyclylalkyl or heterocyclyl group as defined herein. Amido groups, therefore, include, but are not limited to, carbamoyl groups (—C(O)NH₂) and formamide groups (—NHC(O)H). In some embodiments, the amide is —NR³¹C(O)—(C₁₋₅ alkyl) and the group is termed “carbonylamino,” and in others, the amide is —NHC(O)-alkyl and the group is termed “alkanoylamino.”

The term “nitrile” or “cyano” as used herein refers to the —CN group.

The term “amine” (or “amino”) as used herein refers to —NR³⁵R³⁶ groups, wherein R³⁵ and R³⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. The term “alkylamino” is defined as —NR³⁷R³⁸, wherein at least one of R³⁷ and R³⁸ is alkyl and the other is alkyl or hydrogen. The term “arylamino” is defined as —NR³⁹R⁴⁰, wherein at least one of R³⁹ and R⁴⁰ is aryl and the other is aryl or hydrogen.

The term “halogen” or “halo” as used herein refers to bromine, chlorine, fluorine, or iodine. In some embodiments, the halogen is fluorine. In other embodiments, the halogen is chlorine or bromine.

The term “hydroxy' or “hydroxyl”as used herein can refer to —OH or its ionized form, —O.

The term “imide” refers to —C(O)NR⁵⁸C(O)R⁵⁹, wherein R⁵⁸ and R⁵⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR⁶⁰(NR⁶¹) and —N(CR⁶⁰R⁶¹) groups, wherein R⁶⁰ and R⁶¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R⁶⁰ and R⁶¹ are not both simultaneously hydrogen.

The term “nitro” as used herein refers to an —NO₂ group. The term “nitroso” as used herein refers to an —N═O group.

The present technology provides novel NTBI uptake activators, serum-free cell culture media and methods for enhancing iron uptake in a mammalian cell culture. The activators, culture media and methods may also enhance NTBI transfer into cells as part of a serum free cell culture system.

### NTBI Uptake Activators and Methods

The present technology provides a simple method for transporting iron into cells without using transferrin. In particular, the present technology relates generally to novel NTBI uptake activators, cell culture media, methods of enhancing iron uptake in a mammalian cell culture, kits for enhancing iron uptake in cell culture and methods of using the NTBI activators.

The present disclosure provides, inter alia, methods for increasing iron uptake in a mammalian cell culture using non-transferrin-dependent mechanisms. As such, these methods allow for the use of NTBI uptake activators to enhance iron uptake in a mammalian cell culture and transport the iron into the cells. Iron can be delivered to the cells in a number of different ways. Typically, the vast majority of iron is bound to transferrin (Tf) and this Tf-bound iron serves as a major source of iron for the cells. However, in certain conditions where the capacity of plasma Tf to bind iron is surpassed, Non-Transferrin-Bound Iron (NTBI) makes a significant contribution to the cellular iron uptake. Although the mechanism of NTBI transfer into cells remains unclear, it is thought that reduction of ferric iron may be required prior to uptake by a suitable activator. Suitable NTBI activators known in the art include, for example, divalent metal transporter 1 (DMT1), SFT (stimulator of Fe transport), ZIP14 (SLC39A14 zinc transporter) and L-type calcium channels (LTCC) or L-type voltage-dependent calcium channels (LVDCC), etc. However, these activators are associated with drawbacks such as bulk transport of divalent metal ions rather than selective transport of Fe²⁺, transport of Fe³⁺ or Mn²⁺, which is associated with excessive erythrocyte breakdown or hemolysis, increased cell-membrane porosity and obstruction of the Ca²⁺ mediated signaling pathways. Specifically the LTCC or LVDCC type of iron uptake activators also function as Ca²⁻ channel antagonists and, therefore, impede the crucial Ca² mediated signaling pathways.

In one aspect, the present technology provides NTBI uptake activators which enhance the iron uptake of cells cultured in a culture medium. In some embodiments, the culture medium also includes a non-protein bound iron source. In some embodiments, the NTBI uptake activators overcome the above-mentioned drawbacks. In some embodiments, the NTBI uptake activator is not a calcium channel blocker. In some embodiments, the NTBI uptake activators do not impact Tf-mediated iron uptake. In some embodiments, the NTBI uptake activators do not stimulate Fe³⁺ or Mn²⁺ transfer.

In some embodiments, the NTBI uptake activator is a nitrogen heterocycle. In some embodiments, the nitrogen heterocycle is selected from compounds such as pyridine, pyrazine, imidazole, thiazole or bipyridine compounds. These nitrogen heterocycles bind to iron (II) with considerable covalent character rather than form ionic complexes and, therefore, are much more capable of transporting iron across the cell membrane efficiently and quickly with finer control. In some embodiments, the nitrogen heterocycle is a pyridine or pyrazine. In certain embodiments, the pyridine is a substituted pyridine. In an illustrative embodiment, the substituted pyridine is a 2,6 substituted pyridine.

In some embodiments, the NTBI uptake activator is a nitrogen heterocycle, wherein the nitrogen heterocycle is a compound having formula I:

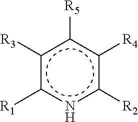

wherein
each of R₁ and R₂ is selected from substituted or unsubstituted alkyl, substituted or unsubstituted aryl, substituted or unsubstituted aralkyl, substituted or unsubstituted alkylamino, substituted or unsubstituted arylamino, substituted or unsubstituted heterocyclyl, and substituted or unsubstituted heteroaryl;
each of R₃, R₄ and R₅ independently represents hydrogen, halogen, hydroxyl, oxo, nitro, nitrile, amino, COOR′, substituted or unsubstituted alkyl, substituted or unsubstituted alkoxy, substituted or unsubstituted aryl, substituted or unsubstituted aralkyl, or substituted or unsubstituted heterocyclyl; and
R′ is a hydrogen or an alkyl group.

In some embodiments, the nitrogen heterocycle is a compound having formula Ia:

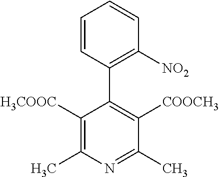

In some embodiments, the nitrogen heterocycle is a compound having formula II:

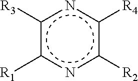

wherein
each of R₁, R₂, R₃ and R₄ independently represents hydrogen, halogen, hydroxyl, oxo, nitro, nitrile, amino, COOR′, substituted or unsubstituted alkyl, substituted or unsubstituted alkoxy, substituted or unsubstituted aryl, substituted or unsubstituted aralkyl, substituted or unsubstituted alkylamino, substituted or unsubstituted arylamino, substituted or unsubstituted heterocyclyl, or substituted or unsubstituted heteroaryl; and
R′ is a hydrogen or an alkyl group.

In some embodiments, the NTBI uptake activators can be used to enhance the iron transport across cell-membrane. In some embodiments, the NTBI uptake activator can be used to enhance iron uptake in a cell culture medium. In some embodiments, the cell culture is a mammalian cell culture. In some embodiments, the NTBI uptake activators are used to transport iron from the culture medium to the cells.

In one aspect, the present disclosure provides a method for enhancing iron uptake in a mammalian cell culture. In some embodiments, the method includes the steps of contacting cells with a culture medium containing a NTBI uptake activator and a non-protein-bound iron source, wherein the NTBI uptake activator is present in an effective amount to enhance the iron uptake of the cells cultured therein compared to cells not contacted with the NTBI uptake activator.

In some embodiments, the NTBI uptake activator includes a nitrogen heterocycle as defined above. The nitrogen activator may be present in an amount sufficient to facilitate the uptake and transfer of NTBI from the culture medium to the cell. In some embodiments, the nitrogen heterocycle is present in the culture medium in a final concentration of about 0.01 μM to about 1 mM. In other embodiments, the nitrogen heterocycle is present in the culture medium in a final concentration of about 0.1 μM to about 500 μM. In an illustrative embodiment, the nitrogen heterocycle is present in the culture medium in a final concentration of about 0.5 μM to about 200 μM. Typically, the nitrogen heterocycle is present in an effective amount to enhance the iron uptake of the cells cultured in the culture medium compared to cells not contacted with the nitrogen heterocycle. In some embodiments, up to three ligands per iron atom are present in the medium.

The nitrogen heretocycles used as NTBI uptake activators in the present technology differ vastly from compounds used in iron chelation therapy. First, unlike the compounds used for iron chelation therapy, the nitrogen heterocycle of the present technology does not form strong chelation complexes with the iron metal but rather forms weaker complexes which can facilitate iron uptake in the culture medium but also enable the iron to be released inside the cell. This is accomplished by suitably substituting the pyridine or pyrazine moiety at the 2- and 6-positions, thereby providing a steric barrier that prevents tight binding to the iron metal. Further, while iron chelators used in chelation therapy have to be extremely water soluble so that they can be given at high loadings and excreted quickly, the iron uptake and transport activators of the present technology have to be slightly hydrophobic to enable passage through the cell membrane. The above defined NTBI uptake activators have been designed with these differences in mind, and suitable nitrogen heterocycles which meet these design criteria can be used in the present methods.

Suitable non-protein-bound iron source added to the culture medium may include one or more Fe²⁺ and/or Fe³⁻ salts. Any suitable iron salt, which is soluble in the culture medium, may be used as the iron source. For example, iron salts selected from ferrous or ferric sulfate, nitrate, ammonium sulfate, chloride, citrate or tartrate may be used as the iron source. In some embodiments, the non-protein-bound iron source is ferric sulfate. In some embodiments, the non-protein-bound iron source is ferrous sulfate. These salts may be used as such or, where necessary, can be used in combination with a chelating agent. In some embodiments, the non-protein-bound iron source is an iron-organic ion chelate. Examples of organic ions include citrate, oxalate, carbonate, formate, malate, tartrate, lactate, succinate, glutamate, fumarate, gluconate and the like. In some embodiments, the iron-organic ion chelate is ferric ammonium citrate. In some embodiments, the chelating agent may be, for example, ethylenediaminetetraacetic acid (EDTA), ethylene glycol-bis(.beta.-aminoethyl ether)-N,N,N′,N′-tetraacetic acid (EGTA), deferoxamine mesylate, dimercaptopropanol, diethylenetriaminepentaacetic acid (DPTA), and trans-1,2-diaminocyclohexane-N,N,N′,N′-tetraacetic acid (CDTA). In an illustrative embodiment, the iron chelate compound used is ferrous sulphate 7H₂O.EDTA (FeSO₄.7H₂O.EDTA), e.g., Sigma F0518, Sigma, St. Louis, Mo.).

The desired concentration of the iron source can be optimized using routine experimentation, depending upon the source used and the quantity of uptake activators in the medium. In some embodiments, the non-protein iron source is present in the culture medium in a final concentration of about 0.01 μM to about 500 μM. In other embodiments, the non-protein iron source is present in the culture medium in a final concentration of about 0.1 μM to about 300 μM. In an illustrative embodiment, the non-protein iron source is present in the culture medium in a final concentration of about 0.4 μM to about 100 μM.

Suitable cell culture media which can be used in the present methods are described in detail later in this specification. The iron uptake activator and the iron source can be added to the same media or to two different culture media. The cell can be contacted with the iron uptake activator and the iron source together, separately or sequentially. When using the same media, the iron uptake activator and the iron source can be added together to a single medium and contacted with the cell or they can be reconstituted separately in the same culture media and contacted with the cell in succession. Thus, in one embodiment, the present disclosure relates to a contacting the cell with a single culture medium containing an activator of iron uptake and a source of iron. In other embodiments, the present disclosure relates to a contacting the cell with a first culture medium containing an activator of iron uptake followed by a second culture medium containing a source of iron.

While mammalian cells are specified in certain embodiments of the present disclosure, any suitable animal cell can be used for the present methods. These cells are described in further detail later in the specification. The mammalian cell used in the present technology can be obtained from any suitable mammalian species, such as human, rodent, hamster or other species by any suitable isolation method. In some embodiments, the cells are human cells. In some embodiments, the human cells are selected from the group consisting of lymphocytes, myeloid cells, monocytes, macrophages, neutrophils, myocytes, fibroblasts, HepG2 carcinoma cells, kidney cells, melanoma cells, and HeLa cells. In other embodiments, the cells are non-human mammalian cells. In an illustrative embodiment, the non-human mammalian cells are Chinese hamster ovary cells.

Suitable iron metal detection techniques known in the art can be employed to detect the presence of iron carried by the NTBI activator and release into the cell. Such methods include fluorescence quenching, inductively coupled plasma atomic emission spectrometry, and light and electron microscopy. In some embodiments, the presence of transferred iron may be detected using the fluorescence quenching technology. Suitable iron-sensitive fluorescent dyes used for this technique include, for example, calcein and phen-green. In some embodiments, iron binding to the calcein fluorophore can be used to detect the fluorescence quenching. For e.g., fluorescence may be detected according to the method described by Espósitol B. P. et. al., in Biochem. Soc. Trans., (2002) 30, (729-732). Calcein is an iron-binding ligand whose fluorescence is quenched upon binding to iron. As a consequence, it has been widely used as a sensor for iron and suggested to be a useful tool for monitoring cytosolic iron and assessing the dynamics of intracellular iron in living cells.

Without being bound to a theory, it is believed that the NTBI uptake activators of the present technology complex with the non-protein bound iron from the culture medium and efficiently transport the iron across the cell membrane. Once inside the cell, the activator readily unloads the iron and makes it available for participation in cellular functions. The NTBI uptake activators function as selective ionophores for the ferrous ion, rather than the ferric iron, and are generally very stable and not susceptible to the medium's redox potential. The iron uptake activators and methods of the present technology are associated with several advantages. The stability and configuration of the present NTBI complexes is such that the iron is transported effectively and made available to participate in cell growth, rather than to catalyze toxic reactions or become non-available to the system. Thus, because the NTBI uptake activators form properly complexed iron, it is available to support cell viability and is essential to the cell culture system. The activators and methods of present technology provide for an increase in the kinetics of iron transport to the cell and reduce the amount of free iron added to the media. Lower amounts of free iron ensure less severe gradients in iron concentration throughout the media and consequently lower risk of poisoning from iron overdose. Further, iron bound by the present nitrogen heterocycle ligands is less likely to oxidize and precipitate out from the solution.

The iron uptake activators and methods of the present technology may be used to transport iron from suitable culture medium to the cells. Suitable cultural media known in the art for use in mammalian cell culture, and available commercially, can be employed in the methods. Typically the cell culture media of the present technology are combinations of salts, energy sources, vitamins, and various indicators, such as pH indicators, for maintaining proper conditions for growth in the media. Iron exists in both the ferrous and ferric state in physiological solutions and undergoes redox cycling depending upon the medium's composition of oxidizing and reducing agents and enzymes, and its exposure to these agents. Therefore, the composition of the culture media is also important since it influences the nature and the stability of the iron complex. The present methods for increasing iron uptake in a mammalian cell culture using non-transferrin-dependent mechanisms such as NTBI uptake activators allow for the production and use of economical serum-free and protein-free media for cell culture. Such serum protein-free media has substantially lower cost and fewer risks than the media containing protein growth factors.

### Cell Culture Media

The present disclosure provides cell culture media and methods that use activators of non-transferrin bound iron (NTBI) uptake in mammalian cells. The culture media and methods may also enhance overall iron transfer into cells as part of a serum free cell culture system. The present disclosure provides, inter alia, methods for increasing iron uptake in a mammalian cell culture using transferrin-dependent and non-transferrin-dependent mechanisms. As such, these methods allow for the production of economical serum-free and/or protein-free media for cell culture.

In one aspect, the present technology provides a media for mammalian cell culture. In some embodiments, the culture medium lacks transferrin. In some embodiments, the culture medium is a serum-free media. In some embodiments, the serum free medium comprises a nitrogen heterocycle and a non-protein-bound iron source, wherein the nitrogen heterocycle is present in an effective amount to enhance the iron uptake of the cells cultured therein compared to cells not contacted with the nitrogen heterocycle. In some embodiments, the nitrogen heterocycle and a non-protein-bound iron are added to a single serum free medium. In other embodiments, the nitrogen heterocycle and a non-protein-bound iron are added to two different serum free media.

Typically, cell culture media formulations are supplemented with a range of additives, including undefined components such as fetal bovine serum (FBS) or extracts from animal embryos, organs or glands. While FBS is the most commonly used supplement in animal cell culture media, other serum sources are also routinely used, including newborn calf, horse and human. These types of chemically undefined supplements serve several useful functions in cell culture media. For example, these supplements provide carriers or chelators for labile or water-insoluble nutrients; bind and neutralize toxic moieties; provide hormones and growth factors, protease inhibitors and essential, often unidentified or undefined low molecular weight nutrients; and protect cells from physical stress and damage. Thus, serum extracts are commonly used as supplements to provide an optimal culture medium for the cultivation of mammalian cells.

Unfortunately, the use of serum or protein additives in tissue culture applications has several drawbacks. For example, the chemical compositions of these supplements and sera vary between lots, even from a single manufacturer. The supplements may also be contaminated with infectious agents (e.g., mycoplasma and viruses) which can seriously undermine the health of the cultured cells and the quality of the final product. The use of undefined components such as serum or animal extracts also prevents the true definition and elucidation of the nutritional and hormonal requirements of the cultured cells, thus eliminating the ability to study, in a controlled way, the effect of specific growth factors or nutrients on cell growth and differentiation in culture. Finally, serum and protein supplementation of culture media can complicate and increase the costs of the purification of the desired substances from the culture media due to nonspecific co-purification of serum or extract proteins.

To overcome these drawbacks of the use of serum or organ/gland extracts, the present disclosure provides media that are specifically formulated to use a non-transferrin bound iron (NTBI) transport pathway. NTBI pathway(s) import iron through low molecular weight chelators, such as citrates, nitrates, or sulfates. As such, chemically defined media can be used that do not rely on the addition of serum or recombinant transferrin.

In various aspects, the present disclosure relates to culture media for use in methods for enhancing iron uptake in cell culture. In one aspect, the present disclosure relates to culture media and methods for cultivating a mammalian cell in vitro. In one embodiment, the methods include replacing protein (particularly animal-derived or recombinant transferrin) in mammalian cell culture media with chemically-defined mixtures. In particular, the disclosure relates to replacing transferrin to media containing such replacements and to compositions comprising mammalian cells in such media. In one embodiment, the present disclosure relates to a single culture medium containing an activator of iron uptake and a source of iron. In other embodiments, the present disclosure relates to a first culture medium containing an activator of iron uptake and a second culture medium containing a source of iron. The present technology also relates to media for suspension culture and to compositions comprising mammalian cells in such suspension culture. Improved levels of recombinant protein expression may be obtained from cells treated with an activator of iron uptake, relative to the level of expression seen in cells grown in medium supplemented with serum.

These culture media and methods allow the replacement of or reduction in the amount of transferrin compared to conventional mammalian culture media with all the associated advantages. Contrasted with naturally derived transferrin (e.g., animal serum), this lowers or eliminates dangers of contamination. Likewise, compared to recombinant transferrin, the present compositions and methods lowers or eliminates the cost for using the recombinant protein. These methods also allow for granular and dynamic control of iron uptake, since process parameters (e.g., amount of inductor, incubation time, temperature, etc.) may be finely controlled.

The culture media may further include one or more ingredients selected from the group of ingredients consisting of one or more amino acids, one or more vitamins, one or more inorganic salts, one or more sugars, one or more buffering salts, and one or more lipids. In one embodiment, the sugar used in the media is D-glucose, while the buffer salt may be N-[2-hydroxyethyl]-piperazine-N′-[2-ethanesulfonic acid] (HEPES). In one embodiment, the culture media may optionally comprise one or more supplements selected from the group of supplements consisting of one or more cytokines, heparin, one or more animal peptides, one or more yeast peptides and one or more plant peptides.

The amino acid ingredients of the present media may include one or more amino acids selected from the group consisting of L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. The vitamin ingredient of the present media may include one or more vitamins selected from the group consisting of biotin, choline chloride, D-Ca²⁺-pantothenate, folic acid, i-inositol, niacinamide, pyridoxine, riboflavin, thiamine and vitamin B₁₂. The inorganic salt ingredient of the present media may include one or more inorganic salts selected from the group consisting of one or more calcium salts, Fe(NO₃)₃, KCl, one or more magnesium salts, one or more manganese salts, NaCl, NaHCO₃, Na₂HPO₄, one or more selenium salts, one or more vanadium salts and one or more zinc salts.

The media may also include the ingredients ethanolamine, D-glucose, N-[2-hydroxyethyl]piperazine-N′-[2-ethanesulfonic acid] (HEPES), insulin, linoleic acid, lipoic acid, phenol red, PLURONIC F68, putrescine, sodium pyruvate, biotin, choline chloride, D-Ca²⁺-pantothenate, folic acid, i-inositol, niacinamide, pyridoxine, riboflavin, thiamine, vitamin B₁₂, one or more calcium salts, Fe(NO₃)₃, KCl, one or more magnesium salts, one or more manganese salts, NaCl, NaHCO₃, Na₂HPO₄, one or more selenium salts, one or more vanadium salts and one or more zinc salts, wherein each ingredient is present in an amount which supports the cultivation of a mammalian cell in vitro.

The specific combinations of the above ingredients and their concentration ranges, in one example of the culture media containing an activator of iron uptake are shown in Table 1.

TABLE 1
Illustrative Cell Culture Media Composition
Containing Activator of Iron Uptake

| | Illustrative Embodiment |
|---|---|
| Component | (mg/L) |
| Ferric sulfate (FeSO4—7H2O) | 0.05 |
| dimethyl 2,6-dimethyl-4-(2- | 0.075 |
| nitrophenyl)pyridine-3,5- | |
| dicarboxylate (NTBI uptake | |
| activator) | |
| Glycine | 7.5 |
| L-Alanine | 8.9 |
| L-Arginine hydrochloride | 211 |
| L-Asparagine-H2O | 15.01 |
| L-Aspartic acid | 13.3 |
| L-Cysteine hydrochloride-H2O | 35.12 |
| L-Glutamic Acid | 14.7 |
| L-Glutamine | 146 |
| L-Histidine hydrochloride-H2O | 21 |
| L-Isoleucine | 4 |
| L-Leucine | 13.1 |
| L-Lysine hydrochloride | 36.5 |
| L-Methionine | 4.5 |
| L-Phenylalanine | 5 |
| L-Proline | 34.5 |
| L-Serine | 10.5 |
| L-Threonine | 11.9 |
| L-Tryptophan | 2.04 |
| L-Tyrosine disodium salt | 7.81 |
| dihydrate | |
| L-Valine | 11.7 |
| Biotin | 0.0073 |
| Choline chloride | 14 |
| D-Calcium pantothenate | 0.5 |
| Folic Acid | 1.3 |
| Niacinamide | 0.036 |
| Pyridoxine hydrochloride | 0.06 |
| Riboflavin | 0.037 |
| Thiamine hydrochloride | 0.3 |
| Vitamin B12 | 1.4 |
| i-Inositol | 18 |
| Calcium Chloride (CaCl2) | 33.22 |
| (anhyd.) | |
| Cupric sulfate (CuSO4—5H2O) | 0.0025 |
| Magnesium Chloride | 57.22 |
| (anhydrous) | |
| Potassium Chloride (KCl) | 223.6 |
| Sodium Bicarbonate | 1176 |
| (NaHCO3) | |
| Sodium Chloride (NaCl) | 7599 |
| Sodium Phosphate dibasic | 142 |
| (Na2HPO4) anhydrous | |
| Zinc sulfate (ZnSO4—7H2O) | 0.863 |
| D-Glucose (Dextrose) | 1802 |
| Hypoxanthine Na | 4.77 |
| Linoleic Acid | 0.084 |
| Lipoic Acid | 0.21 |
| Phenol Red | 1.2 |
| Putrescine 2HCl | 0.161 |
| Sodium Pyruvate | 110 |
| Thymidine | 0.7 |

As will be readily apparent to one of ordinary skill in the art, the concentration of a given ingredient can be increased or decreased beyond the range disclosed and the effect of the increased or decreased concentration can be determined using only routine experimentation. The optimal final concentrations for medium ingredients for culturing particular cell types are typically identified either by empirical studies, in single component titration studies. In single component titration studies using animal cells (e.g., CHO cells or 293 embryonic kidney cells), the concentration of a single medium component is varied while all other constituents and variables are kept constant and the effect of the single component on viability, growth or continued health of the animal cells is measured.

Medium ingredients can be dissolved in a liquid carrier or maintained in dry form. The type of liquid carrier and the method used to dissolve the ingredients into solution vary and may include periodic or continuous mixing, stirring, or shaking, optionally including heating to assist in dissolving the ingredients. In one embodiment, the liquid carrier is water. In another embodiment, the liquid carrier is a buffer, e.g., HEPES or MOPS buffer. In yet another embodiment, the liquid carrier is a concentrated medium lacking one or more components, e.g., an activator of iron uptake and/or an iron source. In one embodiment, the pH of the medium is adjusted to about 7.0-7.6, about 7.1-7.5, or about 7.2-7.4. In one embodiment, the osmolarity of the medium is adjusted to about 260 to about 300 mOsm, about 265 to about 280 mOsm, or about 265 to about 275 mOsm. The type of liquid carrier and the method used to dissolve the ingredients into solution vary and can be determined by one of ordinary skill in the art with no more than routine experimentation. Typically, the medium ingredients can be added in any order.

In some embodiments, the solutions comprising individual ingredients are more concentrated than the concentration of the same ingredients in a 1× media formulation. The ingredients can be 10-fold more concentrated (10× formulation), 25-fold more concentrated (25× formulation), 50-fold more concentrated (50× concentration), or 100-fold more concentrated (100× formulation). More highly concentrated formulations can be made, provided that the ingredients remain soluble and stable.

If the individual medium ingredients are prepared as separate concentrated solutions, an appropriate (sufficient) amount of each concentrate is combined with a diluent to produce a 1× medium formulation. Typically, the diluent used is water, but other solutions, including aqueous buffers, aqueous saline solution, or other aqueous solutions, may be used.

The culture media are typically sterilized to prevent unwanted contamination. Sterilization may be accomplished, for example, by filtration through a low protein-binding membrane filter of about 0.22 μm or 0.45 μm pore size (available commercially from Millipore, Bedford, Mass.) after mixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1× sterile medium formulation. Autoclaving or other elevated temperature-based methods of sterilization are not favored, since many of the components of the present culture media are heat labile and will be irreversibly degraded by temperatures such as those achieved during most heat sterilization methods.

### Use of Culture Media and Methods

In one aspect, the cell culture media may be used to facilitate cultivation of a variety of mammalian cells in suspension or in monolayer cultures. In particular, these media may be used to cultivate mammalian cells or cell lines. Methods for isolation and suspension and monolayer cultivation of a variety of animal cells, including mammalian cells, are known in the art (see, e.g., Freshney, R. I., Culture of Animal Cells: A Manual of Basic Technique, New York: Alan R. Liss, Inc. (1983)) and are described in further detail. While the present media are particularly useful for culturing mammalian cells in suspension, it is to be understood that the media may be used in any standard cell culture protocol whether the cells are grown in suspension, in monolayers, in perfusion cultures (e.g., in hollow fiber microtube perfusion systems), on semi-permeable supports (e.g., filter membranes), in complex multicellular arrays or in any other method by which mammalian cells may be cultivated in vitro.

The media and methods disclosed herein may be used to culture a variety of mammalian cells, including primary epithelial cells (e.g., keratinocytes, cervical epithelial cells, bronchial epithelial cells, tracheal epithelial cells, kidney epithelial cells and retinal epithelial cells) and established cell lines (e.g., 293 embryonic kidney cells, HeLa cervical epithelial cells and PER-C6 retinal cells, MDBK (NBL-1) cells, CRFK cells, MDCK cells, CHO cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, I-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including, but not limited to, heart, liver, kidney, colon, intestine, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃HJIOTI/2 cells, HSDM₁C₃ cells, KLN205 cells, McCoy cells, Mouse L cells, Strain 2071 (Mouse L) cells, L-M strain (Mouse L) cells, L-MTK.sup.-(Mouse L) cells, NCTC clones 2472 and 2555, SCC-PSA1 cells, Swiss/3T3 cells, Indian muntjac cells, SIRC cells, C.sub.II cells, and Jensen cells, or derivatives thereof).

Cells supported by the medium may be derived from any animal, such as a mammal. In one embodiment, the cells are derived from a human. In one embodiment, the cells are mammalian epithelial or fibroblast cells. In illustrative embodiments, the cells are 293 embryonic kidney cells, PER-C6 retinal cells, or CHO cells. The cells cultivated in the present media may be normal cells or abnormal cells (i.e., transformed cells, established cells, or cells derived from diseased tissue samples).

Animal cells for culturing in the media may be obtained commercially, for example, from ATCC (Rockville, Md.), Quantum Biotechnologies (Montreal, Canada) or Invitrogen (San Diego, Calif.). Alternatively, cells may be isolated directly from samples of animal tissue obtained via biopsy, autopsy, donation or other surgical or medical procedure.

In one aspect, the present disclosure provides methods of cultivating cells using the culture medium formulations disclosed herein, comprising: contacting cells with a culture medium containing an effective amount of an activator of iron uptake for a period of time; simultaneous or sequentially adding a source of iron to the medium; and incubating the cells under conditions suitable to allow the growth of the cells in culture. In one embodiment, the cells are incubated in a medium containing an activator of iron uptake and a source of iron separately for at least 30 minutes, at least 60 minutes, at least 2 hours, at least 4 hours, at least 6 hours, at least 8 hours, at least 12 hours, at least 24 hours, at least 48 hours, or longer. In some embodiments, the cells are not incubated in a medium containing an activator of iron uptake and a source of iron separately for longer than about 48 hours or longer than about 24 hours.

The present methods further relate producing a polypeptide, and to polypeptides produced by these methods, the methods comprising: (a) obtaining a mammalian cell, such as a 293 embryonic kidney epithelial cell, PER-C6, and CHO cell, that has been genetically engineered to produce a polypeptide; and (b) contacting the cell with a culture medium including a source of iron and an activator of iron uptake under conditions sufficient to activate an NTBI transport pathway. For example, the polypeptide may be any polypeptide of interest for research or therapeutic purposes.

Optimal methods for genetically engineering a mammalian cell to express a polypeptide of interest are well-known in the art and will, therefore, be familiar to one of ordinary skill. Cells may be genetically engineered prior to cultivation in the media described herein, or they may be transfected with one or more exogenous nucleic acid molecules after being placed into culture in the media. According to one embodiment, genetically engineered cells may be cultivated in the present culture media either as monolayer cultures, or as suspension cultures according to the methods described above. Following cultivation of the cells, the polypeptide of interest may optionally be purified from the cells and/or the used culture medium according to techniques of protein isolation that will be familiar to one of ordinary skill in the art.

The present disclosure further relates to methods of producing a virus, and to viruses produced by these methods, the methods comprising: (a) obtaining a mammalian cell, such as a 293 embryonic kidney epithelial cell, PER-C6, or CHO cell, to be infected with a virus; (b) contacting the cell with a virus under conditions suitable to promote the infection of the cell by the virus; and (c) contacting the cell with a culture medium including a source of iron and an activator of iron uptake under conditions sufficient to activate an NTBI transport pathway. Viruses which may be produced according to these methods include adenoviruses, adeno-associated viruses and retroviruses.

In various embodiments, the cell may be contacted with the virus either prior to, during or following cultivation of the cell in the culture media disclosed herein; optimal methods for infecting a mammalian cell with a virus are known. Virus-infected mammalian cells cultivated in suspension in the media may be expected to produce higher virus titers (e.g., 2-, 3-, 5-, 10-, 20-, 25-, 50-, 100-, 250-, 500-, or 1000-fold higher titers) than those cells not cultivated in suspension in the media. These methods may be used to produce a variety of mammalian viruses and viral vectors, including, but not limited to, adenoviruses, adeno-associated viruses, retroviruses and the like, and are used to produce adenoviruses or adeno-associated viruses. Following cultivation of the infected cells in the present media, the used culture media comprising viruses, viral vectors, viral particles or components thereof (proteins and/or nucleic acids (DNA and/or RNA)) may be used for a variety of purposes, including vaccine production, production of viral vectors for use in cell transfection or gene therapy, infection of animals or cell cultures, study of viral proteins and/or nucleic acids and the like. Alternatively, viruses, viral vectors, viral particles or components thereof may optionally be isolated from the used culture medium according to techniques for protein and/or nucleic acid isolation that will be familiar to one of ordinary skill in the art.

The cell seeding densities can be optimized for the specific culture conditions being used. For routine monolayer culture in plastic culture vessels, an initial seeding density of 1−5×10⁵ cells/cm² may be used, while for suspension cultivation a higher seeding density (e.g., 5−20×10⁵ cells/cm²) may be used.

Mammalian cells are typically cultivated in a cell incubator at about 37° C. The incubator atmosphere should be humidified and should contain about 3-10% carbon dioxide in air, about 8-10% carbon dioxide in air, or about 8% carbon dioxide in air, although cultivation of certain cell lines may require as much as 20% carbon dioxide in air for optimal results.

### Kits

In yet another aspect, a kit for enhancing iron uptake in a mammalian cell culture is provided. In some embodiments, the kit includes a nitrogen heterocycle and a non-protein-bound iron source. Suitable nitrogen heterocycles described above can be included in the kit. In some embodiments of the kit, the nitrogen heterocycle is present in an effective amount to enhance the iron uptake of the cells cultured therein compared to cells not contacted with the nitrogen heterocycle.

The disclosure also provides kits for use in the cultivation of mammalian cells. Kits comprise: one or more containers, wherein a first container contains the first culture medium including an activator of iron uptake; and a second container containing the second culture medium including a source of iron. In some embodiments, the composition of the first culture medium and the second culture medium may be similar. In other embodiments, the composition of the first culture medium and the second culture medium may be different. These kits may further comprise one or more additional containers containing one or more supplements.

Additional kits may include one or more containers wherein a first container contains a basal culture medium prepared as described above and a second container contains an activator of iron uptake. The media in the containers of these kits may be present as dry powders, 1× ready-to-use formulations, or as more concentrated solutions (for example 2×, 5×, 10×, 20×, 25×, 50×, 100×, 500×, 1000× or higher). Additional kits may further comprise one or more additional containers containing one or more supplements selected from the group consisting of one or more cytokines, heparin, one or more peptides, etc.

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described, or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for enhancing iron uptake in a mammalian cell culture, the method comprising:
contacting cells with a culture medium containing a NTBI uptake activator and a non-protein-bound iron source, wherein the NTBI uptake activator is present in an effective amount to enhance the iron uptake of the cells cultured therein compared to cells not contacted with the NTBI uptake activator, and wherein the medium is a serum free medium or the medium lacks transferrin.

2. The method of claim 1, wherein the NTBI uptake activator is a nitrogen heterocycle.

3. The method of claim 2, wherein the nitrogen heterocycle is a pyridine or pyrazine.

4. The method of claim 3, wherein the nitrogen heterocycle is a substituted pyridine.

5. The method of claim 4, wherein the substituted pyridine is a 2,6 substituted pyridine.

6. The method of claim 2, wherein the nitrogen heterocycle is a compound having formula I: 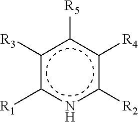
wherein
each of R₁ and R₂ is selected from substituted or unsubstituted alkyl, substituted or unsubstituted aryl, substituted or unsubstituted aralkyl, substituted or unsubstituted alkylamino, substituted or unsubstituted arylamino, substituted or unsubstituted heterocyclyl, and substituted or unsubstituted heteroaryl;
each of R₃, R₄ and R₅ independently represents hydrogen, halogen, hydroxyl, oxo, nitro, nitrile, amino, COOR′, substituted or unsubstituted alkyl, substituted or unsubstituted alkoxy, substituted or unsubstituted aryl, substituted or unsubstituted aralkyl, or substituted or unsubstituted heterocyclyl; and
R′ is a hydrogen or an alkyl group.

7. The method of claim 6, wherein the nitrogen heterocycle is a compound having formula Ia:

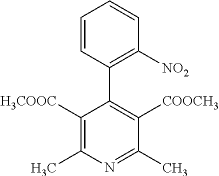

8. The method of claim 2, wherein the nitrogen heterocycle is a compound having formula II: 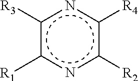
wherein
each of R₁, R₂, R₃ and R₄ independently represents hydrogen, halogen, hydroxyl, oxo, nitro, nitrile, amino, COOR′, substituted or unsubstituted alkyl, substituted or unsubstituted alkoxy, substituted or unsubstituted aryl, substituted or unsubstituted aralkyl, substituted or unsubstituted alkylamino, substituted or unsubstituted arylamino, substituted or unsubstituted heterocyclyl, or substituted or unsubstituted heteroaryl; and
R′ is a hydrogen or an alkyl group.

9. The method of claim 2, wherein the nitrogen heterocycle is present in the culture medium in a final concentration of about 0.5 μM to about 200 μM.

10. The method of claim 1, wherein the non-protein-bound iron source is ferrous sulfate.

11. The method of claim 1, wherein the non-protein-bound iron source is an iron-organic ion chelate.

12. The method of claim 11, wherein the iron-organic ion chelate is ferric ammonium citrate.

13. The method of claim 1, wherein the non-protein iron source is present in the culture medium in a final concentration of about 0.40 μM to about 100 μM.

14. The method of claim 1, wherein the cells are human cells or non-human mammalian cells.

15. The method of claim 14, wherein the human cells are selected from the group consisting of: lyphocytes, myeloid cells, monocytes, macrophages, neutrophils, myocytes, fibroblasts, HepG2 carcinoma cells, kidney cells, melanoma cells, and HeLa cells.

16. The method of claim 14, wherein the non-human mammalian cells are Chinese hamster ovary cells.

17-18. (canceled)

19. A serum free media for mammalian cell culture comprising a nitrogen heterocycle and a non-protein-bound iron source, wherein the nitrogen heterocycle is present in an effective amount to enhance the iron uptake of the cells cultured therein compared to cells not contacted with the nitrogen heterocycle.

20. A kit for enhancing iron uptake in a mammalian cell culture comprising a serum-free or transferrin-free medium, a nitrogen heterocycle and a non-protein-bound iron source, wherein the nitrogen heterocycle is present in an effective amount to enhance the iron uptake of the cells cultured therein compared to cells not contacted with the nitrogen heterocycle.
